(19) United States
(12) Patent Application Publication
(10) Pub. No.: US 2024/0415606 A1
(43) Pub. Date: Dec. 19, 2024

(54) Method, System, and Computer Program Product for Guided and Tracked Cleaning of Medical Devices

(71) Applicants: Vijayakumar Gurusamy, Murrysville, PA (US); Suneil Mandava, Pittsburgh, PA (US)

(72) Inventors: Vijayakumar Gurusamy, Murrysville, PA (US); Suneil Mandava, Pittsburgh, PA (US)

(21) Appl. No.: 18/739,984

(22) Filed: Jun. 11, 2024

Related U.S. Application Data
(60) Provisional application No. 63/508,062, filed on Jun. 14, 2023.

(51) Int. Cl. A61B 90/70; A61L 2/28

(52) U.S. Cl. CPC A61B 90/70; A61L 2/28; A61B 2090/702; A61L 2202/24

(57) ABSTRACT

Described are a method, system, and computer program product for guided and tracked cleaning of medical devices. The method includes determining an item identifier of an item to be cleaned and determining cleaning instructions based on the item identifier. The cleaning instructions include a plurality of steps defining cleaning actions to be performed on the item by a user. The method further includes sequentially presenting each step of the plurality of steps of the cleaning instructions. Sequentially presenting each step of the plurality of steps includes presenting the step via at least one of a visual output and an auditory output of a computing device associated with the user and determining that the step was completed based on an input received at the computing device. The method further includes updating a record associated with the item in at least one database to identify that the item was cleaned.

### CROSS-REFERENCE TO RELATED APPLICATION

This application claims priority to U.S. Provisional Patent Application No. 63/508,062, filed Jun. 14, 2023, the disclosure of which is incorporated by reference herein in its entirety.

### BACKGROUND

### 1. Technical Field

This disclosure relates generally to cleaning medical devices and, in non-limiting embodiments or aspects, to methods, systems, and computer program products for guided and tracked cleaning of medical devices.

### 2. Technical Considerations

A reusable medical device (e.g., endoscope, ventilator, infusion pump, etc.) may require a number of cleaning steps to be performed on the device before the device is safe for reuse. The more complex the medical device, the more difficult it may be for a user to confidently recall the appropriate cleaning steps, as well as execute them properly and in order. For example, a single endoscope may require over two hundred cleaning steps (e.g., disassembling a part, prepping a disinfectant solution, brushing a connective port, rinsing the scope, submerging the scope, etc.). When each of potentially hundreds or thousands of medical devices have different cleaning instructions, it becomes untenable for a user to accurately learn or recall all correct steps in order for a given medical device. A user relying on recall may make mistakes when cleaning a reusable medical device, which creates risk for the patient and personnel who may need to use the reusable medical device next.

Besides following a set of cleaning steps, a user may need to rely on their own subjective review of a medical device to conclude whether the device is sufficiently clean. While it is possible a user could use a confirmatory test, such as a swab culture, confirmatory tests may take a long time to get back results and ensure cleanliness of the medical device. Especially in high-volume hospitals, running slow confirmatory tests is not a viable option. As such, a user may substantially rely on their own quick inspection of a medical device to determine cleanliness, which raises the risk of unclean medical devices being reintroduced to circulation.

There is a need in the art for a technical solution to guide and track cleaning processes for medical devices, so that users do not need to rely on subjectivity and recall to process reusable medical devices. Moreover, there is a need in the art for a technical solution that provides verification of cleaning, to detect whether a cleaning process was properly executed and a medical device is actually clean.

### SUMMARY

Accordingly, provided are improved methods, systems, and computer program products for guided and tracked cleaning of medical devices.

According to non-limiting embodiments or aspects, provided is a computer-implemented method for guided and tracked cleaning of medical devices. The computer-implemented method includes determining, with at least one processor, an item identifier of an item to be cleaned. The method also includes determining, with at least one processor, cleaning instructions based on the item identifier, wherein the cleaning instructions include a plurality of steps defining cleaning actions to be performed on the item by a user. The method further includes sequentially presenting, with at least one processor, each step of the plurality of steps of the cleaning instructions. Sequentially presenting each step of the plurality of steps includes presenting the step via at least one of a visual output and an auditory output of a computing device associated with the user. Sequentially presenting each step of the plurality of steps also includes determining that the step was completed based on an input received at the computing device. The method further includes updating, with at least one processor, a record associated with the item in at least one database to identify that the item was cleaned by the user.

In some non-limiting embodiments or aspects, determining the item identifier may include determining the item identifier based on scan data generated by a scanner associated with the computing device.

In some non-limiting embodiments or aspects, the scanner may include a camera and the scan data may include image data. Determining the item identifier may also include identifying the item based on an output of a machine-learning model given an input of the image data.

In some non-limiting embodiments or aspects, the scanner may include a radio frequency identification (RFID) reader and the scan data may include a RFID identifier of a RFID transmitter positioned on or in the item.

In some non-limiting embodiments or aspects, the method may include determining, with at least one processor, a user identifier of the user based on the scan data generated by the scanner. Updating the record in the at least one database may include associating, in the record, the user identifier with the item identifier and a time that the user completed a final step of the plurality of steps of the cleaning instructions.

In some non-limiting embodiments or aspects, determining the cleaning instructions may include retrieving default instructions associated with the item identifier from the at least one database, wherein the default instructions are predetermined by a manufacturer of the item. Determining the cleaning instructions may also include determining whether additional instructions have been associated with the item identifier in the at least one database that modify the default instructions. Determining the cleaning instructions may further include, in response to determining that the additional instructions have been associated with the item identifier, retrieving the additional instructions associated with the item identifier from the at least one database. The cleaning instructions may include the default instructions modified by the additional instructions.

In some non-limiting embodiments or aspects, determining that the step was completed based on the input received at the computing device may include determining the input based on image data generated by a camera of the computing device. The input may include at least one of the following: a gesture of the user, a condition of the item, a performance of a cleaning action, a physical position of the user, or any combination thereof.

In some non-limiting embodiments or aspects, the input may include a condition of the item. Determining the input may further include determining a measure of cleanliness of the item after the user performs a cleaning action associated with the step of the plurality of steps of the cleaning instructions. The measure of cleanliness may be based on an amount of soiling of the item. Determining the input may further include comparing the measure of cleanliness to a predetermined threshold of cleanliness associated with the step of the plurality of steps of the cleaning instructions. Determining the input may further include, in response to determining that the measure of cleanliness satisfies the predetermined threshold, determining that the step of the plurality of steps of the cleaning instructions was completed.

In some non-limiting embodiments or aspects, determining that the step was completed based on the input received at the computing device may include detecting activation of a foot-controlled input interface associated with the computing device.

In some non-limiting embodiments or aspects, sequentially presenting each step of the plurality of steps of the cleaning instructions may include, in response to determining that the step was completed, updating the record in the at least one database associated with the item to identify the step as a most recently completed step of the cleaning instructions.

According to non-limiting embodiments or aspects, provided is a system for guided and tracked cleaning of medical devices. The system includes at least one processor. The at least one processor is configured to determine an item identifier of an item to be cleaned. The at least one processor is also configured to determine cleaning instructions based on the item identifier. The cleaning instructions include a plurality of steps defining cleaning actions to be performed on the item by a user. The at least one processor is further configured to sequentially present each step of the plurality of steps of the cleaning instructions. When sequentially presenting each step of the plurality of steps, the at least one processor is configured to present the step via at least one of a visual output and an auditory output of a computing device associated with the user, and determine that the step was completed based on an input received at the computing device. The at least one processor is further configured to update a record associated with the item in at least one database to identify that the item was cleaned by the user.

In some non-limiting embodiments or aspects, when determining the item identifier, the at least one processor may be configured to determine the item identifier based on scan data generated by a scanner associated with the computing device.

In some non-limiting embodiments or aspects, the scanner may include a camera and the scan data may include image data. When determining the item identifier, the at least one processor may be configured to identify the item based on an output of a machine-learning model given an input of the image data.

In some non-limiting embodiments or aspects, when determining that the step was completed based on the input received at the computing device, the at least one processor may be configured to determine the input based on image data generated by a camera of the computing device. The input may include at least one of the following: a gesture of the user, a condition of the item, a performance of a cleaning action, a physical position of the user, or any combination thereof.

In some non-limiting embodiments or aspects, the input may include a condition of the item. When determining the input, the at least one processor may be configured to determine a measure of cleanliness of the item after the user performs the cleaning action associated with the step of the plurality of steps of the cleaning instructions. The measure of cleanliness may be based on an amount of soiling of the item. When determining the input, the at least one processor may also be configured to compare the measure of cleanliness to a predetermined threshold of cleanliness associated with the step of the plurality of steps of the cleaning instructions. When determining the input, the at least one processor may further be configured to, in response to determining that the measure of cleanliness satisfies the predetermined threshold, determine that the step of the plurality of steps of the cleaning instructions was completed.

According to non-limiting embodiments or aspects, provided is a computer program product for guided and tracked cleaning of medical devices. The computer program product includes at least one non-transitory computer-readable medium including one or more instructions that, when executed by at least one processor, cause the at least one processor to determine an item identifier of an item to be cleaned. The one or more instructions also cause the at least one processor to determine cleaning instructions based on the item identifier. The cleaning instructions include a plurality of steps defining cleaning actions to be performed on the item by a user. The one or more instructions also cause the at least one processor to sequentially present each step of the plurality of steps of the cleaning instructions. When sequentially presenting each step of the plurality of steps, the at least one processor is configured to present the step via at least one of a visual output and an auditory output of a computing device associated with the user, and determine that the step was completed based on an input received at the computing device. The one or more instructions further cause the at least one processor to update a record associated with the item in at least one database to identify that the item was cleaned by the user.

In some non-limiting embodiments or aspects, the one or more instructions that cause the at least one processor to determine the item identifier may cause the at least one processor to determine the item identifier based on scan data generated by a scanner associated with the computing device.

In some non-limiting embodiments or aspects, the scanner may include a camera and the scan data may include image data. The one or more instructions that cause the at least one processor to determine the item identifier may cause the at least one processor to identify the item based on an output of a machine-learning model given an input of the image data.

In some non-limiting embodiments or aspects, the one or more instructions that cause the at least one processor to determine that the step was completed based on the input received at the computing device may cause the at least one processor to determine the input based on image data generated by a camera of the computing device. The input may include at least one of the following: a gesture of the user, a condition of the item, a performance of a cleaning action, a physical position of the user, or any combination thereof.

In some non-limiting embodiments or aspects, the input may include a condition of the item. The one or more instructions that cause the at least one processor to determine the input may cause the at least one processor to determine a measure of cleanliness of the item after the user performs a cleaning action associated with the step of the plurality of steps of the cleaning instructions. The measure of cleanliness may be based on an amount of soiling of the item. The one or more instructions that cause the at least one processor to determine the input may also cause the at least one processor to compare the measure of cleanliness to a predetermined threshold of cleanliness associated with the step of the plurality of steps of the cleaning instructions. The one or more instructions that cause the at least one processor to determine the input may further cause the at least one processor to, in response to determining that the measure of cleanliness satisfies the predetermined threshold, determine that the step of the plurality of steps of the cleaning instructions was completed.

Further non-limiting embodiments or aspects are set forth in the following numbered clauses:

Clause 1: A computer-implemented method comprising: determining, with at least one processor, an item identifier of an item to be cleaned; determining, with at least one processor, cleaning instructions based on the item identifier, wherein the cleaning instructions comprise a plurality of steps defining cleaning actions to be performed on the item by a user; sequentially presenting, with at least one processor, each step of the plurality of steps of the cleaning instructions, wherein sequentially presenting each step of the plurality of steps comprises: presenting the step via at least one of a visual output and an auditory output of a computing device associated with the user; and determining that the step was completed based on an input received at the computing device; and updating, with at least one processor, a record associated with the item in at least one database to identify that the item was cleaned by the user.

Clause 2: The computer-implemented method of clause 1, wherein determining the item identifier comprises: determining the item identifier based on scan data generated by a scanner associated with the computing device.

Clause 3: The computer-implemented method of clause 1 or clause 2, wherein the scanner comprises a camera, the scan data comprises image data, and determining the item identifier further comprises: identifying the item based on an output of a machine-learning model given an input of the image data.

Clause 4: The computer-implemented method of any of clauses 1-3, wherein the scanner comprises a radio frequency identification (RFID) reader and the scan data comprises a RFID identifier of a RFID transmitter positioned on or in the item.

Clause 5: The computer-implemented method of any of clauses 1-4, further comprising: determining, with at least one processor, a user identifier of the user based on the scan data generated by the scanner; and wherein updating the record in the at least one database comprises: associating, in the record, the user identifier with the item identifier and a time that the user completed a final step of the plurality of steps of the cleaning instructions.

Clause 6: The computer-implemented method of any of clauses 1-5, wherein determining the cleaning instructions comprises: retrieving default instructions associated with the item identifier from the at least one database, wherein the default instructions are predetermined by a manufacturer of the item; determining whether additional instructions have been associated with the item identifier in the at least one database that modify the default instructions; and in response to determining that the additional instructions have been associated with the item identifier, retrieving the additional instructions associated with the item identifier from the at least one database, wherein the cleaning instructions comprise the default instructions modified by the additional instructions.

Clause 7: The computer-implemented method of any of clauses 1-6, wherein determining that the step was completed based on the input received at the computing device comprises: determining the input based on image data generated by a camera of the computing device, wherein the input comprises at least one of the following: a gesture of the user, a condition of the item, a performance of a cleaning action, a physical position of the user, or any combination thereof.

Clause 8: The computer-implemented method of any of clauses 1-7, wherein the input comprises a condition of the item, and wherein determining the input further comprises: determining a measure of cleanliness of the item after the user performs the cleaning action associated with the step of the plurality of steps of the cleaning instructions, wherein the measure of cleanliness is based on an amount of soiling of the item; comparing the measure of cleanliness to a predetermined threshold of cleanliness associated with the step of the plurality of steps of the cleaning instructions; and in response to determining that the measure of cleanliness satisfies the predetermined threshold, determining that the step of the plurality of steps of the cleaning instructions was completed.

Clause 9: The computer-implemented method of any of clauses 1-8, wherein determining that the step was completed based on the input received at the computing device comprises: detecting activation of a foot-controlled input interface associated with the computing device.

Clause 10: The computer-implemented method of any of clauses 1-9, wherein sequentially presenting each step of the plurality of steps of the cleaning instructions further comprises: in response to determining that the step was completed, updating the record in the at least one database associated with the item to identify the step as a most recently completed step of the cleaning instructions.

Clause 11: A system comprising: at least one processor configured to: determine an item identifier of an item to be cleaned; determine cleaning instructions based on the item identifier, wherein the cleaning instructions comprise a plurality of steps defining cleaning actions to be performed on the item by a user; sequentially present each step of the plurality of steps of the cleaning instructions, wherein, when sequentially presenting each step of the plurality of steps, the at least one processor is configured to: present the step via at least one of a visual output and an auditory output of a computing device associated with the user; and determine that the step was completed based on an input received at the computing device; and update a record associated with the item in at least one database to identify that the item was cleaned by the user.

Clause 12: The system of clause 11, wherein, when determining the item identifier, the at least one processor is configured to: determine the item identifier based on scan data generated by a scanner associated with the computing device.

Clause 13: The system of clause 11 or clause 12, wherein the scanner comprises a camera, the scan data comprises image data, and when determining the item identifier, the at least one processor is configured to: identify the item based on an output of a machine-learning model given an input of the image data.

Clause 14: The system of any of clauses 11-13, wherein, when determining that the step was completed based on the input received at the computing device, the at least one processor is configured to: determine the input based on image data generated by a camera of the computing device, wherein the input comprises at least one of the following: a gesture of the user, a condition of the item, a performance of a cleaning action, a physical position of the user, or any combination thereof.

Clause 15: The system of any of clauses 11-14, wherein the input comprises a condition of the item, and wherein, when determining the input, the at least one processor is configured to: determine a measure of cleanliness of the item after the user performs a cleaning action associated with the step of the plurality of steps of the cleaning instructions, wherein the measure of cleanliness is based on an amount of soiling of the item; compare the measure of cleanliness to a predetermined threshold of cleanliness associated with the step of the plurality of steps of the cleaning instructions; and in response to determining that the measure of cleanliness satisfies the predetermined threshold, determine that the step of the plurality of steps of the cleaning instructions was completed.

Clause 16: A computer program product comprising at least one non-transitory computer-readable medium comprising one or more instructions that, when executed by at least one processor, cause the at least one processor to: determine an item identifier of an item to be cleaned; determine cleaning instructions based on the item identifier, wherein the cleaning instructions comprise a plurality of steps defining cleaning actions to be performed on the item by a user; sequentially present each step of the plurality of steps of the cleaning instructions, wherein, when sequentially presenting each step of the plurality of steps, the at least one processor is configured to: present the step via at least one of a visual output and an auditory output of a computing device associated with the user; and determine that the step was completed based on an input received at the computing device; and update a record associated with the item in at least one database to identify that the item was cleaned by the user.

Clause 17: The computer program product of clause 16, wherein the one or more instructions that cause the at least one processor to determine the item identifier cause the at least one processor to: determine the item identifier based on scan data generated by a scanner associated with the computing device.

Clause 18: The computer program product of clause 16 or clause 17, wherein the scanner comprises a camera, the scan data comprises image data, and the one or more instructions that cause the at least one processor to determine the item identifier cause the at least one processor to: identify the item based on an output of a machine-learning model given an input of the image data.

Clause 19: The computer program product of any of clauses 16-18, wherein the one or more instructions that cause the at least one processor to determine that the step was completed based on the input received at the computing device cause the at least one processor to: determine the input based on image data generated by a camera of the computing device, wherein the input comprises at least one of the following: a gesture of the user, a condition of the item, a performance of a cleaning action, a physical position of the user, or any combination thereof.

Clause 20: The computer program product of any of clauses 16-19, wherein the input comprises a condition of the item, and wherein the one or more instructions that cause the at least one processor to determine the input cause the at least one processor to: determine a measure of cleanliness of the item after the user performs a cleaning action associated with the step of the plurality of steps of the cleaning instructions, wherein the measure of cleanliness is based on an amount of soiling of the item; compare the measure of cleanliness to a predetermined threshold of cleanliness associated with the step of the plurality of steps of the cleaning instructions; and in response to determining that the measure of cleanliness satisfies the predetermined threshold, determine that the step of the plurality of steps of the cleaning instructions was completed.

These and other features and characteristics of the present disclosure, as well as the methods of operation and functions of the related elements of structures and the combination of parts and economics of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosed subject matter.

### BRIEF DESCRIPTION OF THE DRAWINGS

Additional advantages and details are explained in greater detail below with reference to the non-limiting, exemplary embodiments that are illustrated in the accompanying schematic figures, in which:

FIG. 1 is a schematic diagram of a system for guided and tracked cleaning of medical devices, according to some non-limiting embodiments or aspects;

FIG. 2 is a schematic diagram of a system for guided and tracked cleaning of medical devices, according to some non-limiting embodiments or aspects;

FIG. 3 is a schematic diagram of example components of one or more devices of FIG. 1 and FIG. 2, according to some non-limiting embodiments or aspects;

FIG. 4 is a flow diagram of a method for guided and tracked cleaning of medical devices, according to some non-limiting embodiments or aspects; and

FIG. 5 is a flow diagram of a method for guided and tracked cleaning of medical devices, according to some non-limiting embodiments or aspects.

### DETAILED DESCRIPTION

For purposes of the description hereinafter, the terms “end”, “upper”, “lower”, “right”, “left”, “vertical”, “horizontal”, “top”, “bottom”, “lateral”, “longitudinal,” and derivatives thereof shall relate to non-limiting embodiments or aspects as they are oriented in the drawing figures. However, it is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects of the disclosed subject matter. Hence, specific dimensions and other physical characteristics related to the embodiments or aspects disclosed herein are not to be considered as limiting.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. In addition, reference to an action being “based on” a condition may refer to the action being “in response to” the condition. For example, the phrases “based on” and “in response to” may, in some non-limiting embodiments or aspects, refer to a condition for automatically triggering an action (e.g., a specific operation of an electronic device, such as a computing device, a processor, and/or the like).

As used herein, the term “communication” may refer to the reception, receipt, transmission, transfer, provision, and/or the like of data (e.g., information, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices. A computing device may also be a desktop computer or other form of non-mobile computer.

As used herein, the term “server” may refer to or include one or more computing devices that are operated by or facilitate communication and processing for multiple parties in a network environment, such as the Internet, although it will be appreciated that communication may be facilitated over one or more public or private network environments and that various other arrangements are possible. Further, multiple computing devices (e.g., servers, desktop computers, mobile devices, etc.) directly or indirectly communicating in the network environment may constitute a “system.”

As used herein, the term “system” may refer to one or more computing devices or combinations of computing devices (e.g., processors, servers, client devices, software applications, components of such, and/or the like). Reference to “a device,” “a server,” “a processor,” and/or the like, as used herein, may refer to a previously-recited device, server, or processor that is recited as performing a previous step or function, a different device, server, or processor, and/or a combination of devices, servers, and/or processors. For example, as used in the specification and the claims, a first device, a first server, or a first processor that is recited as performing a first step or a first function may refer to the same or different device, server, or processor recited as performing a second step or a second function.

The methods, systems, and computer program products described herein provide numerous technical advantages in systems for guided and tracked cleaning of medical devices. First, described systems and methods reduce error in medical device cleaning by automatically and correctly identifying a medical device to be cleaned and the appropriate cleaning instructions associated with the medical device. Described systems and methods can differentiate between different models of the same category of medical device and identify the cleaning instructions specific to a model of medical device. Described systems and methods further promote the safe sanitation of medical devices and reduce user error by guiding users through cleaning instructions sequentially step-by-step (e.g., visual and/or auditory feedback). Furthermore, described systems and methods may automatically document compliance with predetermined cleaning instructions, by updating records associated with the medical device in a database without prompting by user, so that logs can be kept of cleaning events, user identities, and times of activity. Such automatic documentation allows for auditability in the case of a disease transmission event, and reduces the likelihood a medical device will be requested for use if the medical device has not been documented as properly cleaned.

In some non-limiting embodiments or aspects, further technical advantages are provided where a computing device that is used to present the cleaning instructions is associated with a scanner (e.g., camera), which may automatically determine when each step is completed, without the user having to physically touch an input of the computing device. For example, by using machine vision (e.g., image-data based machine-learning models) to verify that cleaning steps are being performed- and to even verify when a medical device is sufficiently cleaned-a user need not touch a computer keyboard or screen to indicate compliance. Instead, a user may use a gesture, body positioning, and/or image of the device itself to show that a step or process is complete. This reduces transmission of bacteria in either direction between the keyboard and medical device. In some non-limiting embodiments or aspects, the user may be provided with a foot-controlled input (e.g., one or more foot pedal buttons), so that the user may direct the computing device (e.g., to go to the next step or a previous step) without touching the computing device with their hands.

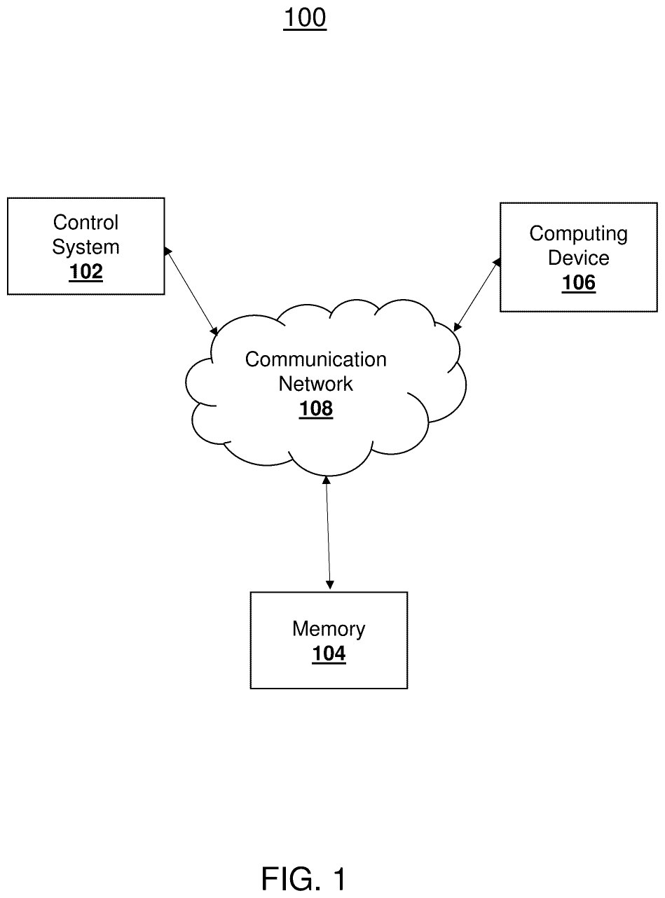

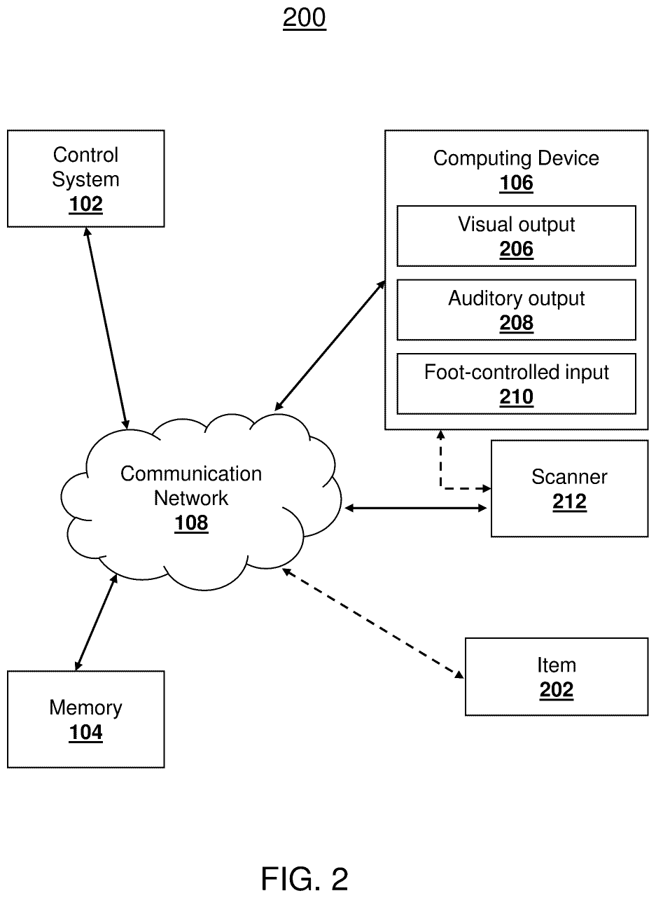

Referring now to FIGS. 1 and 2, FIGS. 1 and 2 are a schematic diagram of example system 100 and system 200 in which devices, systems, and/or methods, described herein, may be implemented. As shown in FIG. 1, system 100 may include control system 102, memory 104, computing device 106, and communication network 108. As shown in FIG. 2, system 200 may include the devices and systems of system 100, as well as item 202 and scanner 212. Control system 102, memory 104, computing device 106, item 202, and scanner 212 may interconnect (e.g., establish a connection to communicate) via wired connections, wireless connections, or a combination of wired and wireless connections.

With specific reference to FIGS. 1 and 2, control system 102 may include one or more computing devices configured to communicate with memory 104, computing device 106, and/or item 202 at least partly over communication network 108. Control system 102 may be configured to receive item and cleaning process data from computing device 106, determine appropriate cleaning instructions for an item (e.g., medical device) to be cleaned (e.g., item 202), and guide and track the cleaning process for the item. Control system 102 may include or be in communication with memory 104. Control system 102 may include or be included in the same system as computing device 106. Control system 102 may be positioned remotely from computing device 106, such as in a remote computational cluster. Control system 102 may provide guidance and tracking functionality for one or more computing devices 106 at one or more locations (e.g., hospitals, rooms, departments, etc.).

With specific reference to FIGS. 1 and 2, memory 104 may include one or more computing devices configured to communicate with control system 102 and/or computing device 106 at least partly over communication network 108. Memory 104 may be configured to store data associated with cleaning instructions for a plurality of items (e.g., a plurality of item 202). Memory 104 may store default instructions for cleaning the plurality of items as predetermined by a manufacturer of each item. Memory 104 may further store additional instructions for cleaning items as input by users associated with a hospital or medical facility. Final cleaning instructions may include the default instructions in combination with (e.g., modified by) the additional instructions. Memory 104 may further store at least one record of at least one item that is stored at a hospital (e.g., item 202). Each item record may include, but is not limited to, an item identifier (e.g., a number, code, string, description, etc., that identifies an item), an item serial number, an item model/type identifier, a default instructions identifier (e.g., a number, code, string, description, etc., that identifies a default set of instructions), an additional instructions identifier (e.g., a number, code, string, description, etc., that identifies an additional set of instructions), a last user identifier (e.g., a number, code, string, description, etc., that identifies a user who last used an associated item), a time of last cleaning (e.g., a date, time, time period, etc., during which an associated item was last cleaned), a last completed cleaning step (e.g., a number, code, string, description, etc., that identifies a step of a cleaning instruction that was most recently completed), a time of last completed cleaning step (e.g., a date, time, time period, etc., during which an associated last completed cleaning step was performed), an item status (e.g., a number code, string, description, etc., that identifies a status of the item associated with a level of cleanliness and/or state relative to cleanliness, such as “Cleaned”, “Ready for Use”, “Reprocessing Needed”, “Contaminated”, “Cleaning in Progress”, “15% Processed”, etc.), a patient identifier (e.g., a number, code, string, description, etc., that identifies a patient for which an item was or may be used), a procedure identifier (e.g., a number code, string, description, etc., that identifies a procedure for which an item was or may be used), a location identifier (e.g., a number, code, string, description, etc., that identifies a location of last detection, such as in a hospital location, room, or department), and/or the like. Memory 104 may communicate with and/or be included in control system 102.

With specific reference to FIGS. 1 and 2, computing device 106 may include one or more processors that are configured to communicate with control system 102, memory 104, and/or item 202 at least partly over communication network 108. Computing device 106 may be associated with a user and may include at least one user interface for presenting cleaning instructions and receiving user input. Computing device 106 may include visual output 206 (e.g., a display screen, indicator lights, projector) for displaying cleaning steps (e.g., images, video, text), auditory output 208 (e.g., a speaker, an analog beeper or chime, etc.) for narrating and guiding cleaning steps, and/or the like (as shown in FIG. 2). Computing device 106 may include and/or be associated with at least one type of scanner 212 (e.g., a camera, a radio frequency identification (RFID) reader, a barcode scanner, etc.) for automatically identifying an item to be cleaned (e.g., using machine vision to identify an item, scanning a RFID transmitter in or on the item, scanning a barcode printed on the item, etc.) and/or identifying a user performing the cleaning (e.g., using machine vision to identify personnel, scanning a RFID transmitter in a user badge, scanning a barcode on a user identification (ID), etc.) (as shown in FIG. 2). Computing device 106 may further include foot-controlled input 210 (e.g., one or more foot pedal buttons) acting as an input component of computing device 106 (as shown in FIG. 2). In some non-limiting embodiments or aspects, computing device 106 may be a mobile device on a stand. In some non-limiting embodiments or aspects, computing device 106 may be a desktop or laptop computer with an associated webcam (e.g., acting at least partly as scanner 212).

With specific reference to FIGS. 1 and 2, communication network 108 may include one or more wired and/or wireless networks over which the systems and devices of system 100 may communicate. For example, communication network 108 may include a cellular network (e.g., a long-term evolution (LTE®) network, a third generation (3G) network, a fourth generation (4G) network, a fifth generation (5G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of these or other types of networks.

With specific reference to FIG. 2, scanner 212 may include and/or be associated with one computing devices configured to communicate with control system 102, computing device 106, and/or item 202 at least partly over communication network 108. Scanner 212 may include one or more sensors configured to collect scan data (e.g., scanned information, signals, etc.) associated with item 202, a user cleaning item 202, and/or the like. In some non-limiting embodiments or aspects, scanner 212 may include a camera, and the scan data generated by scanner 212 may include image data (e.g., including one or more frames of two- or three-dimensional image capture). Additionally or alternatively, scanner 212 may include a radio frequency identification (RFID) reader, and the scan data generated by scanner 212 may include an RFID identifier (e.g., a number, a code, a string, a description, etc.) of a RFID transmitter positioned on or in item 202. The RFID transmitter may include a one-way or two-way RFID transceiver (e.g., an RFID tag) attached to or embedded in item 202. Scanner 212 may be included in or associated with computing device 106. For example, scanner 212 may include, at least partly, a webcam of a desktop or laptop computer of computing device 106. By way of further example, scanner 212 may include, at least partly, a barcode scanner and/or a high-frequency (HR) RFID reader connected to an input port (e.g., a universal serial bus (USB)) of computing device 106.

With specific reference to FIG. 2, item 202 may include and/or be associated with one or more computing devices configured to communicate with control system 102, computing device 106, and/or scanner 212 at least partly over communication network 108. Item 202 may include any device, component, or accessory that is reusable from one patient-related procedure to another and which may require cleaning between procedural uses. In some non-limiting embodiments or aspects, item 202 may include or be associated with non-electronic devices, such as surgical instruments (e.g., scalpels, forceps, scissors, clamps, etc.), suction tips and tubes, dental instruments (e.g., dental mirrors, probes, forceps, etc.), reusable orthopedic implants (e.g., surgical devices for temporary fixation), speculums, anesthesia equipment (e.g., masks, tubing, etc.), sterilization trays and containers, and/or the like. In some non-limiting embodiments or aspects, item 202 may include or be associated with electronic devices, such as endoscopes, electrosurgical units (ESUs), laparoscopic instruments (e.g., cameras, light sources, etc.), defibrillators, ultrasound probes, patient monitors (e.g., sensor leads, blood pressure cuffs, oxygen sensors, etc.), infusion pumps, ventilators, and/or the like. Item 202 may include or have attached an identification component, such as an RFID transmitter, a bar code (e.g., a one-dimensional barcode, a two-dimensional barcode, etc.), a part number, and/or the like.

The number and arrangement of systems and devices shown in FIGS. 1 and 2 are provided as an example. There may be additional systems and/or devices, fewer systems and/or devices, different systems and/or devices, or differently arranged systems and/or devices than those shown in FIG. 1. Furthermore, two or more systems or devices shown in FIG. 1 may be implemented within a single system or device, or a single system or device shown in FIG. 1 may be implemented as multiple, distributed systems or devices. Additionally or alternatively, a set of systems (e.g., one or more systems) or a set of devices (e.g., one or more devices) of system 100 may perform one or more functions described as being performed by another set of systems or another set of devices of system 100.

With further reference to FIGS. 1 and 2, in some non-limiting embodiments or aspects, control system 102 may be configured to perform one or more steps of a method for guiding and tracking cleaning of medical devices. For example, control system 102 may determine an item identifier (e.g., code, serial number, text string, etc.) of an item (e.g., reusable medical device) to be cleaned (e.g., dissembled, washed, soaked, sterilized, autoclaved, etc.) (e.g., item 202). Control system 102 may determine cleaning instructions (e.g., a plurality of steps defining one or more cleaning actions to be performed) based on the item identifier. For example, control system 102 may determine the item identifier based on scan data (e.g., image data, RFID data, barcode data) generated by a scanner (e.g., camera, RFID receiver, barcode scanner, etc.) associated with computing device 106 (e.g., scanner 212), and control system 102 may retrieve cleaning instructions from the database (e.g., memory 104) based on the item identifier. By way of further example, control system 102 may use a camera as the scanner to capture image data of the item and may identify the item based on an output of a machine-learning model (e.g., a model trained on images of medical devices) given an input of the image data.

When determining the cleaning instructions, control system 102 may retrieve default instructions (e.g., a minimum plurality of cleaning steps) associated with the item identifier from the at least one database (e.g., memory 104). The default instructions may be predetermined and input by a manufacturer of the item. In some non-limiting embodiments or aspects, each step of cleaning instructions may include a brief textual description of cleaning steps to be performed, along with one or more optional supporting data files that may be provided (e.g., displayed, played, etc.) to a user, such as an image file depicting the cleaning step, a video file depicting the cleaning step, an audio file describing the cleaning step, and/or the like. Control system 102 may further determine whether additional and/or alternative instructions have been associated with the item identifier in the at least one database that modify and/or replace one or more steps of the default instructions. For example, a hospital staff member may provide input of one or more enhanced or additional cleaning steps to augment the default cleaning instructions. In response to determining that additional and/or alternative instructions have been associated with the item identifier, control system 102 may retrieve the additional and/or alternative instructions associated with the item identifier from the at least one database. The final cleaning instructions as determined by the control system 102 may be a combination of the default instructions and additional instructions (e.g., the default instructions modified by the inclusion of additional instructions). Additionally or alternatively, the final cleaning instructions as determined by the control system 102 may be one of the default instructions with one or more steps replaced by the one or more alternative instructions.

In some non-limiting embodiments or aspects, control system 102 and/or computing device 106 may sequentially present each step of the plurality of steps of the cleaning instructions to a user (e.g., clinician) performing the cleaning. In doing so, control system 102 and/or computing device 106 may present each step via at least one of a visual output (e.g., image, video, and/or text on a display screen, such as via visual output 206) and an auditory output (e.g., narrated instructions through a speaker, such as via auditory output 208) of computing device 106 associated with the user. To that end, control system 102 may cause computing device 106 to sequentially present each step by transmitting data (e.g., including text, data from one or more image files, data from one or more video files, data from one or more audio files, etc.) to computing device 106 for display and/or audibilization.

In some non-limiting embodiments or aspects, control system 102 may determine that each step is completed based on an input received at computing device 106. For example, when determining that each step is completed, control system 102 may determine the input based on image data generated by a camera of computing device 106. Control system 102 and/or computing device 106 may receive input, analyze the input, and provide output to the user via computing device 106 at least partly in real-time (e.g., immediately or substantially immediately, such that an action is taken in direct response to an event that is occurring at the time, such as on the scale of milliseconds, seconds, etc.). The input may include, but is not limited to, at least one of the following: a gesture of the user (e.g., a hand wave to indicate “next step”, a specific hand sign or number of fingers raised, a combination of arm and hand position, etc.); a performance of a cleaning action (e.g., as determined by video analysis); a physical position of the user (e.g., standing toward the side of the field of view to indicate “next step”); or any combination thereof. When the input includes at least a condition of the item such as a measure of cleanliness of the item, control system 102 may determine the measure of cleanliness of the item after the user performs a cleaning action. The measure of cleanliness may be based on an amount of soiling of the item (e.g., percent of surface area covered by filth, which may be determined by color, texture, patterns, brightness, etc.). Control system 102 may compare the measure of cleanliness to a predetermined threshold of cleanliness (e.g., a threshold percent of surface area covered by filth) associated with the cleaning step. In response to determining that the measure of cleanliness satisfies (e.g., meets or exceeds) the predetermined threshold, control system 102 may determine that the cleaning step is completed. Additionally or alternatively, control system 102 may detect activation of a user-controlled input, such as a foot-controlled input interface (e.g., a foot pedal button, such as foot-controlled input 210), associated with computing device 106 to determine that a cleaning step is completed.

In some non-limiting embodiments or aspects, control system 102 may update a record (e.g., a data log, stored item entry, etc.) associated with the item in at least one database (e.g., memory 104) to identify that the item was cleaned by the user. Control system 102 may determine a user identifier (e.g., badge number, user ID, employee name, etc.) of the user based on scan data generated by a scanner (e.g., camera, RFID receiver, barcode scanner, etc.) associated with computing device 106. For example, a user may wear a badge that may include or may be associated with a scannable device, such a barcode, a user identification (ID) number, a RFID tag, and/or the like. Control system 102 may update the record in the at least one database (e.g., memory 104) by associating the user identifier with the item identifier. For example, the user's badge may be scanned proximal in time and/or location with an item that is detected by computing device 106 and/or scanner 212, and the user and item may be associated with one another in memory 104. Control system 102 may also associate the user identifier and the item identifier, in the record, with a time that the user completed a final step of the cleaning instructions for the item. Additionally or alternatively, when control system 102 is sequentially presenting each step of a set of cleaning instructions for an item being cleaned, and as each step is completed, control system 102 may update the record to identify the latest completed step, the time of the latest completed step, the user that performed the latest completed step, a status of the item as of the latest completed step, and/or the like.

In some non-limiting embodiments or aspects, the described systems may include locally executed software on computing device 106, which may control a display screen to provide information to a user. The computing device 106 may include a scanner (e.g., scanner 212) to identify the reusable medical device (e.g., item 202), such as by scanning an RFID transmitter, a barcode, an image of the item, and/or the like. The described systems and methods may automatically identify the reusable item with the granularity of detail to include manufacturer, catalog number, model, version, serial number, and/or the like. Such information may be included directly in an RFID identifier code, barcode, or other on-item indicia, or through a lookup system. Additionally or alternatively, computing device 106 may employ a camera with artificial intelligence-based machine vision to automatically identify the item.

In some non-limiting embodiments or aspects, the described systems may have manufacturer “instructions for use” (IFUs) (e.g., the default cleaning instructions) programmed into the system (e.g., stored in memory 104), which may be updated as manufacturers make updates and input changes to the IFUs. After identifying a reusable item, the described systems and method may look up an associated IFU and provide the IFU, in a step-by-step fashion, to an end user (e.g., a clinician) on a display screen in the room (e.g., computing device 106). As the user completes each step, the user may input into the system that they have completed the step. For example, the user may click a button on the display, provide a gesture, tap a foot pedal, or provide another method of feedback to indicate they have completed the cleaning step. After completing the cleaning step, the described system may document the time of completion of the completed step and display the next step in the cleaning instructions for the reusable item. The described system may also have the option to provide information on the reusable item and cleaning steps through an auditory method, so that the user may have hands free to handle the reusable item.

In some non-limiting embodiments or aspects, the described systems and methods may enable hospitals to program additional steps for the cleaning process and/or edit the default cleaning process for each item. This may be because the hospital's infection control has established different processes than the manufacturer's default guidelines. It may also be because the hospital has equipment that the manufacturer did not include in the default guidelines. For example, a hospital or trade group may recommend drying the inside lumen of a flexible endoscope after completing sanitization. Drying the inside lumen is not included in most scope manufacturers' IFUs, but an additional step of drying the inside lumen could be included as additional instructions that would guide a user to follow one or more additional steps.

In some non-limiting embodiments or aspects, the described systems and methods may identify the user by scanning the user's badge, by prompting the user to enter a user identifier, and/or the like. Doing so may allow the user to be identified without a manual login process. The described systems may also identify the user by using machine vision (e.g., facial recognition and comparison with a database of authorized users). In a cleaning environment, a user's badge may be under scrubs or cleaning clothes and may be hard to access. Hands may also be covered in gloves, which may make data entry difficult. Accordingly, the automatic identification of the user may eliminate touches of the display, badge, and other surfaces.

In some non-limiting embodiments or aspects, the described systems and methods may automatically detect (e.g., using machine vision) whether the current step in the cleaning process is complete. For example, a camera of computing device 106 may capture image data of an endoscope being placed in a soak bath. Control system 102 and/or computing device 106 may use the image data to automatically identify the item, identify the user cleaning the item, identify the step of placing the item in a soak bath, and document some or all of such data in one or more records of a database (e.g., memory 104). The user would not need to enter this information into a checklist or touch a display. These techniques reduce or eliminate the number of user touches, which would reduce the likelihood of bacteria being transferred from one surface to another. These techniques would also speed up the work for the user and reduce the number of footsteps for the user, as cleaning rooms may be quite large and cleaning equipment may be far away from each other.

In some non-limiting embodiments or aspects, the described system may scan the reusable items during various steps of the cleaning process to detect the cleanliness (e.g., level of soiling) of the reusable item. Thresholds of cleanliness may be set for various steps to make sure that each step meets cleaning standards. For example, an item being cleaned may come into the cleaning room at a 78% clean level (e.g., as determined by machine vision inspecting a percent coverage of the outer surface with soiling, such as by lubricant, biological material, etc.). The item may be placed into a soak bath and, after emerging, be determined to be at a 94% clean level. Certain steps may require the item to reach a certain threshold level of cleanliness (e.g., 95%) before allowing the item to progress to the next step of cleaning. This may be because certain cleaning machines require a certain volume of soiling to be removed before placing the item in the cleaning machine. Currently, determining sufficient cleanliness for placement in a cleaning machine is up to the subjective visual inspection of a user, which is variable between users. The described systems and methods provide an objective evaluation of cleanliness and verification process to ensure that the surfaces of an item are sufficiently clean and dry.

Provided below with reference to the foregoing figures is a descriptive illustration of non-limiting embodiments or aspects of the present disclosure. Consider a user (e.g., a clinician) that needs to prepare an endoscope (e.g., item 202) for use in a patient operation. The user may begin by going to a storage container (e.g., a drying cabinet) currently holding the endoscope and removing the endoscope from the storage container. An item identifier (e.g., provided by a RFID transmitter) may be on or in the endoscope and may be read by a scanner (e.g., RFID reader) associated with the storage container. Control system 102 may update a record of the item identifier (e.g., in memory 104) to note that the user (e.g., by updating a user identifier in the record) removed the endoscope at a specific time (e.g., by updating a time field of the record). The user may bring the endoscope to computing device 106 (e.g., a desktop computer) that has a forward-facing camera and an RFID reader, and the user may stand in front of computing device 106 and present the endoscope in view of the camera.

With further reference to the foregoing illustration, control system 102 may use the camera of computing device 106 to identify the endoscope (e.g., by inputting one or more images from the camera into an image classification machine-learning model trained on a set of images of items in a hospital inventory). Additionally or alternatively, control system 102 may identify the endoscope using the RFID reader to read an item identifier from the RFID transmitter positioned on or in the endoscope. Control system 102 may then retrieve a record of the item from a database (e.g., memory 104), based on a lookup performed using the item identifier. Control system 102 may determine, based on a log of events associated with the record of the item, that the endoscope was recently removed from its storage container by the user. Control system 102 may also search the log of events associated with the record of the item and determine if any cleaning steps have already been performed on the endoscope. In the present example, control system 102 may determine that while the endoscope was previously cleaned and placed in a drying cabinet, an amount of time has elapsed (e.g., seven days) since the time of last cleaning that necessitates the endoscope being reprocessed for use (e.g., determined by comparing a current time to a time of last recorded completion of a final step of cleaning instructions, and checking a field of the record associated with allowable time between cleanings). As part of that determination, control system 102 may retrieve default instructions for cleaning the endoscope based on predetermined manufacturer cleaning instructions associated with the endoscope's item record. Control system 102 may further search for additional instructions that may have been programmed by hospital personnel, to require additional or modified cleaning steps beyond the default instructions.

With further reference to the foregoing illustration, control system 102 may display for a user on a display screen of computing device 106 (or cause to be displayed by transmitting instructions), a current cleaning step. The current cleaning step may be determined by control system 102 based on the default instructions, modified by the additional instructions, in comparison to prior-completed cleaning steps of the endoscope (if any). For example, control system 102 may first determine the default instructions for the endoscope, which may specify the following steps: (1) wiping debris off the exterior of the endoscope with a detergent solution; (2) clearing channels with air and detergent solution; (3) separating all detachable parts of the endoscope; (4) cleaning the external surface of the endoscope with detergent using cloths, sponges, and/or brushes; (5) flushing all accessible channels with detergent and sterile water; (6) placing the endoscope in an ultrasonic cleaning bath; (7) rinsing the endoscope with sterile water to remove detergent; (8) soaking the endoscope in a high-level disinfectant solution; (9) rinsing the endoscope with sterile water to remove disinfectant; (10) rinsing surface and channels with air and ethyl or isopropyl alcohol solution (e.g., 70% to 90% alcohol); (11) reassembling any detachable parts of the endoscope; and (12) storing the endoscope in a safe, dry, sterile environment (e.g., drying cabinet). It will be appreciated that the foregoing are exemplary cleaning steps of a set of cleaning instructions, and regulatory bodies may specify additional or different cleaning steps based on specific medical devices.

In some non-limiting embodiments or aspects, control system 102 may determine final cleaning instructions based on additional instructions that modify the default instructions (e.g., to modify, add, or replace cleaning steps of the default instructions). For example, hospital personnel may have programmed additional instructions that specify, after step (10) of the default instructions for the endoscope, that the user should take a sample culture (e.g., swab) of an exterior surface and an interior channel of the endoscope, and test the sample culture to assure there is no remaining microbiological material. The culture test of the additional instructions may become step (11) of the final cleaning instructions, while step (11) of the default instructions may become step (12), and step (12) of the default instructions may become step (13).

In some non-limiting embodiments or aspects, control system 102 may determine that steps (1)-(7) do not need to be repeated, but since sterilization needs to occur, the user should start at step (8) of the final cleaning instructions (e.g., based on the default instructions modified by the additional instructions). As such, control system 102 may cause the display screen of computing device 106 to display step (8), to instruct the user to soak the endoscope in a high-level disinfectant solution. User may then follow the on-screen instructions. The camera of computing device 106 may use machine vision to monitor the user and endoscope, and automatically determine that user placed the endoscope in the disinfectant bath for the requisite amount of time, thereby completing step (8). Additionally or alternatively, user may operate a foot-pedal button of computing device 106 to indicate to control system 102 when step (8) is completed. Upon receiving the feedback that step (8) is completed, control system 102 may update the display screen of computing device 106 to display step (9) of the final cleaning instructions. User may then continue to complete each step in the same manner, until the final step of the cleaning instructions is complete. As each step is completed, and through completion of the final step, control system 102 may update a record associated with the item to reflect the last step completed, when the last step was completed, and which user completed the last step. In this way, the user is guided through the cleaning instructions for the item in a process that reduces user error and reduces the number of user touch-interactions with computing device 106 that might cause contamination of the endoscope.

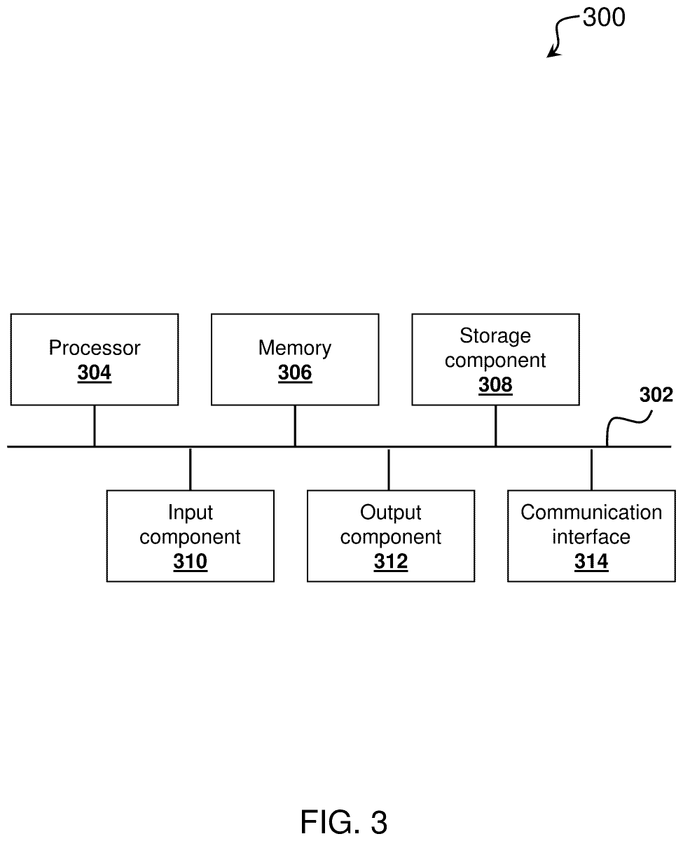

Referring now to FIG. 3, shown is a diagram of example components of device 300, according to some non-limiting embodiments. Device 300 may correspond to one or more devices of control system 102, memory 104, computing device 106, communication network 108, scanner 212, and/or item 202, as an example. In some non-limiting embodiments or aspects, such systems or devices may include at least one device 300 and/or at least one component of device 300. The number and arrangement of components shown are provided as an example. In some non-limiting embodiments, device 300 may include additional components, fewer components, different components, or differently arranged components than those shown. Additionally, or alternatively, a set of components (e.g., one or more components) of device 300 may perform one or more functions described as being performed by another set of components of device 300.

As shown in FIG. 3, device 300 may include bus 302, processor 304, memory 306, storage component 308, input component 310, output component 312, and communication interface 314. Bus 302 may include a component that permits communication among the components of device 300. In some non-limiting embodiments or aspects, processor 304 may be implemented in hardware, firmware, or a combination of hardware and software. For example, processor 304 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 306 may include random access memory (RAM), read only memory (ROM), and/or another type of dynamic or static storage device (e.g., flash memory, magnetic memory, optical memory, etc.) that stores information and/or instructions for use by processor 304.

With continued reference to FIG. 3, storage component 308 may store information and/or software related to the operation and use of device 300. For example, storage component 308 may include a hard disk (e.g., a magnetic disk, an optical disk, a magneto-optic disk, a solid state disk, etc.) and/or another type of computer-readable medium. Input component 310 may include a component that permits device 300 to receive information, such as via user input (e.g., a touch screen display, a keyboard, a keypad, a mouse, a button, a switch, a microphone, etc.). Additionally, or alternatively, input component 310 may include a sensor for sensing information (e.g., a global positioning system (GPS) component, an accelerometer, a gyroscope, an actuator, etc.). Output component 312 may include a component that provides output information from device 300 (e.g., a display, a speaker, one or more light-emitting diodes (LEDs), etc.). Communication interface 314 may include a transceiver-like component (e.g., a transceiver, a separate receiver and transmitter, etc.) that enables device 300 to communicate with other devices, such as via a wired connection, a wireless connection, or a combination of wired and wireless connections. Communication interface 314 may permit device 300 to receive information from another device and/or provide information to another device. For example, communication interface 314 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 300 may perform one or more processes described herein. Device 300 may perform these processes based on processor 304 executing software instructions stored by a computer-readable medium, such as memory 306 and/or storage component 308. A computer-readable medium may include any non-transitory memory device. A memory device includes memory space located inside of a single physical storage device or memory space spread across multiple physical storage devices. Software instructions may be read into memory 306 and/or storage component 308 from another computer-readable medium or from another device via communication interface 314. When executed, software instructions stored in memory 306 and/or storage component 308 may cause processor 304 to perform one or more processes described herein. Additionally, or alternatively, hardwired circuitry may be used in place of or in combination with software instructions to perform one or more processes described herein. Thus, embodiments described herein are not limited to any specific combination of hardware circuitry and software. The term “configured to,” as used herein, may refer to an arrangement of software, device(s), and/or hardware for performing and/or enabling one or more functions (e.g., actions, processes, steps of a process, and/or the like). For example, “a processor configured to” may refer to a processor that executes software instructions (e.g., program code) that cause the processor to perform one or more functions.

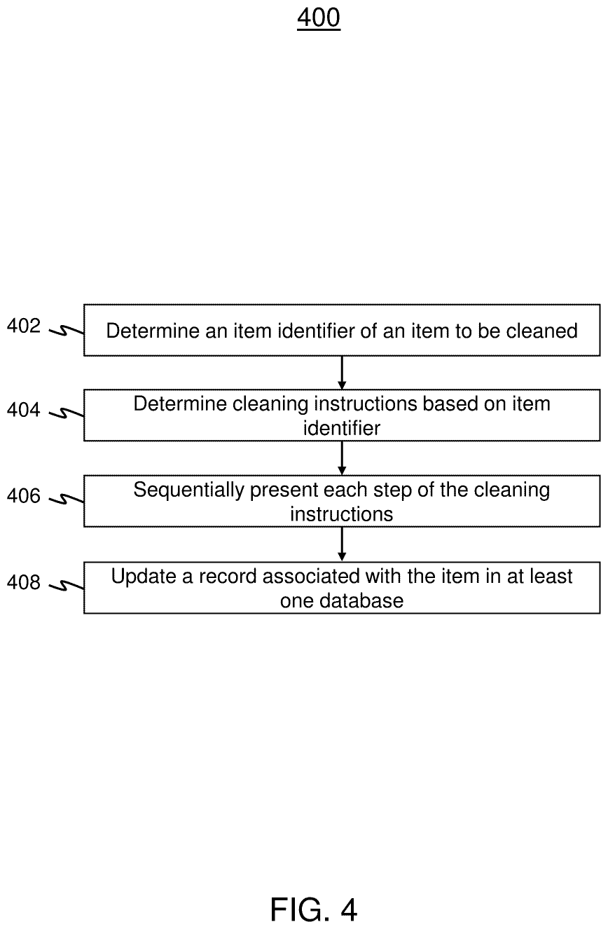

Referring now to FIG. 4, shown is a flow diagram of a process 400 for guided and tracked cleaning of medical devices, according to some non-limiting embodiments or aspects. The steps shown in FIG. 4 are for example purposes only. It will be appreciated that additional, fewer, different, and/or a different order of steps may be used in non-limiting embodiments or aspects. In some non-limiting embodiments or aspects, one or more of the steps of process 400 may be performed (e.g., completely, partially, and/or the like) by control system 102, scanner 212, and/or computing device 106. In some non-limiting embodiments or aspects, one or more of the steps of process 400 may be performed (e.g., completely, partially, and/or the like) by another system, another device, another group of systems, or another group of devices, separate from or including control system 102, scanner 212, and/or computing device 106. In some non-limiting embodiments or aspects, a step may be automatically performed in response to performance and/or completion of a prior step.

As shown in FIG. 4, at step 402, process 400 may include determining an item identifier of an item to be cleaned. For example, control system 102 may determine an item identifier of an item (e.g., reusable medical device) to be cleaned (e.g., item 202).

In some non-limiting embodiments or aspects, when determining the item identifier (at step 402), control system 102 may determine the item identifier based on scan data generated by a scanner associated with the computing device 106 (e.g., scanner 212). The scanner may include a camera and the scan data may include image data. In some non-limiting embodiments or aspects, when determining the item identifier (at step 402), control system 102 may identify the item based on an output of a machine-learning model (e.g., operated on a computing device associated with control system 102 and/or computing device 106) given an input of the image data. The scanner may also include a RFID reader and the scan data may include a RFID identifier of a RFID transmitter positioned on or in the item being cleaned.

As shown in FIG. 4, at step 404, process 400 may include determining cleaning instructions based on the item identifier. For example, control system 102 may determine cleaning instructions based on the item identifier. The cleaning instructions may include a plurality of steps defining cleaning actions to be performed on the item by a user, and stored data associated with the cleaning instructions (e.g., in memory 104) may include text data, image data, video data, audio data, and/or the like.

In some non-limiting embodiments or aspects, when determining the cleaning instructions (at step 404), control system 102 may retrieve default instructions associated with the item identifier from the at least one database (e.g., memory 104). The default instructions may be predetermined by a manufacturer of the item (e.g., based on one or more tables storing item identifiers in association with manufacturer identifiers, and storing manufacturer identifiers in association with identifiers of default cleaning instructions). Control system 102 may further determine whether additional instructions have been associated with the item identifier in the at least one database that modify the default instructions (e.g., based on one or more tables storing item identifiers in association with identifiers of additional instructions). In response to determining that the additional instructions have been associated with the item identifier, control system 102 may retrieve the additional instructions associated with the item identifier from the at least one database. The final cleaning instructions determined by control system 102 may include the default instructions modified by the additional instructions (e.g., replacing one or more steps of the default instructions with one or more steps the additional instructions, inserting one or more steps into the default instructions with one or more steps of the additional instructions, and/or the like).

As shown in FIG. 4, at step 406, process 400 may include sequentially presenting each step of the cleaning instructions. For example, control system 102 and/or computing device 106 may sequentially present each step of the plurality of steps of the cleaning instructions. By way of further example, control system 102 may cause computing device 106 to sequentially present each step of the plurality of steps of the cleaning instructions. Sequentially presenting each step of the plurality of steps may include presenting the step via visual output and/or auditory output of computing device 106 associated with the user, and determining that the step was completed based on an input received at computing device 106. For example, computing device 106 may display, in a user interface of a display screen of computing device 106, text data associated with the current cleaning step. Additionally or alternatively, computing device 106 may display, in a user interface of a display screen of computing device 106 (e.g., of visual output 206), image data and/or video data associated with the current cleaning step. Additionally or alternatively, computing device 106 may audibilize, with a speaker of computing device 106 (e.g., of auditory output 208), audio data associated with the current cleaning step.

In some non-limiting embodiments or aspects, when determining that the step was completed based on the input received at the computing device 106 (at step 406), control system 102 may determine the input based on image data generated by a camera of computing device 106. The input may include at least one of the following: a gesture of the user, a condition of the item, a performance of a cleaning action, a physical position of the user, or any combination thereof. In some non-limiting embodiments or aspects, the input may include a condition of the item. When determining the input (at step 406), control system 102 may determine a measure of cleanliness of the item after the user performs a cleaning action associated with the step of the plurality of steps of the cleaning instructions. The measure of cleanliness may be based on an amount of soiling of the item. Control system 102 may further compare the measure of cleanliness to a predetermined threshold of cleanliness associated with the step of the plurality of steps of the cleaning instructions and, in response to determining that the measure of cleanliness satisfies the predetermined threshold, determine that the step of the plurality of steps of the cleaning instructions was completed.

In some non-limiting embodiments or aspects, when sequentially presenting each step of the plurality of steps of the cleaning instructions (at step 406), control system 102 may, in response to determining that the step was completed, update the record in the at least one database associated with the item to identify the step as a most recently completed step of the cleaning instructions.

In some non-limiting embodiments or aspects, when determining that the step was completed based on the input received at the computing device 106 (at step 406), control system 102 and/or computing device 106 may detect activation of a foot-controlled input interface associated with computing device 106 (e.g., foot-controlled input 210).

As shown in FIG. 4, at step 408, process 400 may include updating a record associated with the item in at least one database. For example, control system 102 may update a record associated with the item in at least one database (e.g., memory 104) to identify that the item was cleaned by the user.

In some non-limiting embodiments or aspects, control system 102 may further determine a user identifier based on the scan data generated by the scanner associated with computing device 106. In such cases, when updating the record (at step 408), control system 102 may associate, in the record, the user identifier with the item identifier. Additionally or alternatively, control system 102 may associate the user identifier with the item identifier and a time that the user completed a final step of the plurality of steps of the cleaning instructions.

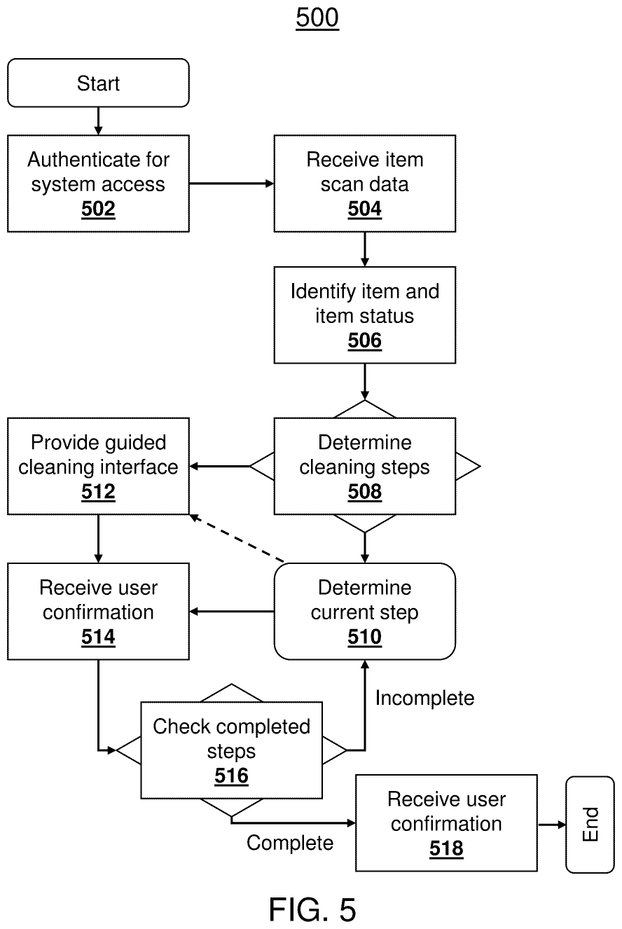

Referring now to FIG. 5, shown is a flow diagram of a process 500 for guided and tracked cleaning of medical devices, according to some non-limiting embodiments or aspects. The steps shown in FIG. 5 are for example purposes only. It will be appreciated that additional, fewer, different, and/or a different order of steps may be used in non-limiting embodiments or aspects. In some non-limiting embodiments or aspects, one or more of the steps of process 500 may be performed (e.g., completely, partially, and/or the like) by control system 102, scanner 212, and/or computing device 106. In some non-limiting embodiments or aspects, one or more of the steps of process 500 may be performed (e.g., completely, partially, and/or the like) by another system, another device, another group of systems, or another group of devices, separate from or including control system 102, scanner 212, and/or computing device 106. In some non-limiting embodiments or aspects, a step may be automatically performed in response to performance and/or completion of a prior step.

As shown in FIG. 5, process 500 may include, at step 502, authenticating a user for access to the cleaning system. For example, control system 102 and/or computing device 106 may determine and authenticate an identity of the user by scanning a badge of the user with scanner 212, such as to read a barcode, user ID number, an RFID tag, and/or the like. Additionally or alternatively, control system 102 and/or computing device 106 may determine and authenticate an identity of the user by scanning the user's face with scanner 212, for authentication by machine vision.

As shown in FIG. 5, process 500 may include, at step 504, receiving scan data of the item to be cleaned (e.g., item 202). For example, in response to or in parallel with the user being identified, control system 102 and/or computing device 106 may scan the item to be cleaned using scanner 212. By way of further example, the scan data may include image data from a camera, video data from a camera, barcode data from a barcode, an RFID identifier from an RFID transmitter, and/or the like.

As shown in FIG. 5, process 500 may include, at step 506, identifying the item and a status of the item. For example, in response to receiving the scan data, control system 102 and/or computing device 106 may determine the item that was scanned, such as by looking up a record of the item in memory 104 based on the scan data. By way of further example, information to be used for looking up the item in memory 104 may be contained in the scan data or further determined by inputting the scan data to a machine learning model that is trained to identify a stamped part number, the shape of the item, and/or the like. The status of the item may be determined directly or indirectly from one or more fields of a record associated with the scanned item, and the status may include a status relative to cleanliness, a last cleaning step performed, a time of last cleaning step, a time of last full cleaning, and/or the like.

As shown in FIG. 5, process 500 may include, at step 508, determining cleaning steps associated with the scanned item. For example, based on an item identifier determined from the scan data, control system 102 and/or computing device 106 may load a set of default cleaning instructions associated with the item identifier. The default cleaning instructions may be modified, at least partly, based on additional cleaning instructions that are also saved in association with the item identifier. The final cleaning instructions, including at least the default cleaning instructions and any additional cleaning instructions, may be loaded into memory of computing device 106 for step-by-step presentation to the user.

As shown in FIG. 5, process 500 may include, at step 510, determining a current cleaning step of the cleaning instructions. For example, control system 102 and/or computing device 106 may determine a current cleaning step of the cleaning instructions. In some non-limiting embodiments or aspects, if no steps of the cleaning instructions were previously performed, then control system 102 may determine the first step as the current step. In some non-limiting embodiments or aspects, if certain steps of the cleaning instructions were not previously performed but are not necessary (e.g., as determined based on time since last cleaning, image analysis of scanned item, etc.), then control system 102 and/or computing device 106 may determine a current step after the unnecessary steps. In some non-limiting embodiments or aspects, if the item was already partly cleaned according to the final cleaning instructions, control system 102 and/or computing device 106 may determine a current cleaning step based on the last completed cleaning step (e.g., the step occurring immediately thereafter).

As shown in FIG. 5, process 500 may include, at step 512, providing a guided cleaning interface for user to follow along with the cleaning instructions. For example, control system 102 and/or computing device 106 may display, via a display of computing device 106 (e.g., a display screen, projector, etc.), the current cleaning step of the cleaning instructions. The cleaning instructions may be represented by displayed text data, displayed image data, displayed video data, and/or audibilized audio data. The current cleaning step may be updated to the next cleaning step after control system 102 and/or computing device 106 determine that the current cleaning step has been completed (e.g., based on image data received from a camera of computing device 106, based on user input, and/or the like).

As shown in FIG. 5, process 500 may include, at step 514, receiving user confirmation of completion of a cleaning step. For example, control system 102 and/or computing device 106 may receive user confirmation of completion of a cleaning step via user input provided to computing device 106. By way of further example, the user may activate a foot-controlled input (e.g., foot-controlled input 210) to send a signal to computing device 106 indicating that the current cleaning step has been completed.

As shown in FIG. 5, process 500 may include, at step 516, checking the completed steps of the cleaning instructions. For example, control system 102 and/or computing device 106 may check the list of steps in cleaning instructions to determine which steps have been completed and/or which steps have yet to be completed. If there are steps of the cleaning instructions remaining (e.g., cleaning is “incomplete”), process 500 may proceed back to steps 510 and 512. If there are no steps of the cleaning instructions remaining (e.g., cleaning is “complete”), process 500 may proceed to step 518. With each completed step, control system 102 and/or computing device 106 may update a record of the item in memory 104 to indicate the last completed step, time of last completed step, and user that performed the last completed step.

As shown in FIG. 5, process 500 may include, at step 518, receiving user confirmation of completion of the cleaning instructions. For example, control system 102 and/or computing device 106 may determine that all steps of the cleaning instructions have been performed and may indicate completion to the user. The user may then input to computing device 106 an acknowledgement and/or confirmation that the cleaning instructions have been completed. Upon receiving user confirmation, control system 102 may exit the guided cleaning routine and may update a record of the item in memory 104, such as to indicate the last completed step, the time of last completed step, the time of last completed cleaning, the user that performed the last completed step, and the user that performed the last completed cleaning. The user may then proceed to send the item for use, store the cleaned item, and/or fetch a new item to be cleaned, which may begin process 500 again. When a user is cleaning multiple items, step 502 may or may not be required in between items that are cleaned, and user may be required to reauthenticate themselves if too much time has passed between items (e.g., 30 seconds, 1 minute, 5 minutes, etc.).

Although the disclosure has been described in detail for the purpose of illustration, it is to be understood that such detail is solely for that purpose and that the disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

What is claimed is:

1. A computer-implemented method comprising:
determining, with at least one processor, an item identifier of an item to be cleaned;
determining, with at least one processor, cleaning instructions based on the item identifier, wherein the cleaning instructions comprise a plurality of steps defining cleaning actions to be performed on the item by a user;
sequentially presenting, with at least one processor, each step of the plurality of steps of the cleaning instructions, wherein sequentially presenting each step of the plurality of steps comprises:
presenting the step via at least one of a visual output and an auditory output of a computing device associated with the user; and
determining that the step was completed based on an input received at the computing device; and
updating, with at least one processor, a record associated with the item in at least one database to identify that the item was cleaned by the user.

2. The computer-implemented method of claim 1, wherein determining the item identifier comprises:
determining the item identifier based on scan data generated by a scanner associated with the computing device.

3. The computer-implemented method of claim 2, wherein the scanner comprises a camera, the scan data comprises image data, and determining the item identifier further comprises:
identifying the item based on an output of a machine-learning model given an input of the image data.

4. The computer-implemented method of claim 2, wherein the scanner comprises a radio frequency identification (RFID) reader and the scan data comprises a RFID identifier of a RFID transmitter positioned on or in the item.

5. The computer-implemented method of claim 2, further comprising:
determining, with at least one processor, a user identifier of the user based on the scan data generated by the scanner; and
wherein updating the record in the at least one database comprises:
associating, in the record, the user identifier with the item identifier and a time that the user completed a final step of the plurality of steps of the cleaning instructions.

6. The computer-implemented method of claim 1, wherein determining the cleaning instructions comprises:
retrieving default instructions associated with the item identifier from the at least one database, wherein the default instructions are predetermined by a manufacturer of the item;
determining whether additional instructions have been associated with the item identifier in the at least one database that modify the default instructions; and
in response to determining that the additional instructions have been associated with the item identifier, retrieving the additional instructions associated with the item identifier from the at least one database, wherein the cleaning instructions comprise the default instructions modified by the additional instructions.

7. The computer-implemented method of claim 1, wherein determining that the step was completed based on the input received at the computing device comprises:
determining the input based on image data generated by a camera of the computing device, wherein the input comprises at least one of the following: a gesture of the user, a condition of the item, a performance of a cleaning action, a physical position of the user, or any combination thereof.

8. The computer-implemented method of claim 7, wherein the input comprises a condition of the item, and wherein determining the input further comprises:
determining a measure of cleanliness of the item after the user performs the cleaning action associated with the step of the plurality of steps of the cleaning instructions, wherein the measure of cleanliness is based on an amount of soiling of the item;
comparing the measure of cleanliness to a predetermined threshold of cleanliness associated with the step of the plurality of steps of the cleaning instructions; and
in response to determining that the measure of cleanliness satisfies the predetermined threshold, determining that the step of the plurality of steps of the cleaning instructions was completed.

9. The computer-implemented method of claim 1, wherein determining that the step was completed based on the input received at the computing device comprises:
detecting activation of a foot-controlled input interface associated with the computing device.

10. The computer-implemented method of claim 1, wherein sequentially presenting each step of the plurality of steps of the cleaning instructions further comprises:
in response to determining that the step was completed, updating the record in the at least one database associated with the item to identify the step as a most recently completed step of the cleaning instructions.

11. A system comprising:
at least one processor configured to:
determine an item identifier of an item to be cleaned;
determine cleaning instructions based on the item identifier, wherein the cleaning instructions comprise a plurality of steps defining cleaning actions to be performed on the item by a user;
sequentially present each step of the plurality of steps of the cleaning instructions, wherein, when sequentially presenting each step of the plurality of steps, the at least one processor is configured to:
present the step via at least one of a visual output and an auditory output of a computing device associated with the user; and
determine that the step was completed based on an input received at the computing device; and
update a record associated with the item in at least one database to identify that the item was cleaned by the user.

12. The system of claim 11, wherein, when determining the item identifier, the at least one processor is configured to:
determine the item identifier based on scan data generated by a scanner associated with the computing device.

13. The system of claim 12, wherein the scanner comprises a camera, the scan data comprises image data, and when determining the item identifier, the at least one processor is configured to:
identify the item based on an output of a machine-learning model given an input of the image data.

14. The system of claim 11, wherein, when determining that the step was completed based on the input received at the computing device, the at least one processor is configured to:
determine the input based on image data generated by a camera of the computing device, wherein the input comprises at least one of the following: a gesture of the user, a condition of the item, a performance of a cleaning action, a physical position of the user, or any combination thereof.

15. The system of claim 14, wherein the input comprises a condition of the item, and wherein, when determining the input, the at least one processor is configured to:
determine a measure of cleanliness of the item after the user performs a cleaning action associated with the step of the plurality of steps of the cleaning instructions, wherein the measure of cleanliness is based on an amount of soiling of the item;
compare the measure of cleanliness to a predetermined threshold of cleanliness associated with the step of the plurality of steps of the cleaning instructions; and
in response to determining that the measure of cleanliness satisfies the predetermined threshold, determine that the step of the plurality of steps of the cleaning instructions was completed.

16. A computer program product comprising at least one non-transitory computer-readable medium comprising one or more instructions that, when executed by at least one processor, cause the at least one processor to:
determine an item identifier of an item to be cleaned;
determine cleaning instructions based on the item identifier, wherein the cleaning instructions comprise a plurality of steps defining cleaning actions to be performed on the item by a user;
sequentially present each step of the plurality of steps of the cleaning instructions, wherein, when sequentially presenting each step of the plurality of steps, the at least one processor is configured to:
present the step via at least one of a visual output and an auditory output of a computing device associated with the user; and
determine that the step was completed based on an input received at the computing device; and
update a record associated with the item in at least one database to identify that the item was cleaned by the user.

17. The computer program product of claim 16, wherein the one or more instructions that cause the at least one processor to determine the item identifier cause the at least one processor to:
determine the item identifier based on scan data generated by a scanner associated with the computing device.

18. The computer program product of claim 17, wherein the scanner comprises a camera, the scan data comprises image data, and the one or more instructions that cause the at least one processor to determine the item identifier cause the at least one processor to:
identify the item based on an output of a machine-learning model given an input of the image data.

19. The computer program product of claim 16, wherein the one or more instructions that cause the at least one processor to determine that the step was completed based on the input received at the computing device cause the at least one processor to:
determine the input based on image data generated by a camera of the computing device, wherein the input comprises at least one of the following: a gesture of the user, a condition of the item, a performance of a cleaning action, a physical position of the user, or any combination thereof.

20. The computer program product of claim 19, wherein the input comprises a condition of the item, and wherein the one or more instructions that cause the at least one processor to determine the input cause the at least one processor to:
determine a measure of cleanliness of the item after the user performs a cleaning action associated with the step of the plurality of steps of the cleaning instructions, wherein the measure of cleanliness is based on an amount of soiling of the item;
compare the measure of cleanliness to a predetermined threshold of cleanliness associated with the step of the plurality of steps of the cleaning instructions; and
in response to determining that the measure of cleanliness satisfies the predetermined threshold, determine that the step of the plurality of steps of the cleaning instructions was completed.
